(19) United States
(12) Patent Application Publication
(10) Pub. No.: US 2003/0157708 A1
(43) Pub. Date: Aug. 21, 2003

(54) Cell stimulation apparatus and a cell stimulation method

(75) Inventor: Miwako Ozaki, Wako-shi (JP)

Correspondence Address: GREENBLUM & BERNSTEIN, P.L.C., 1950 ROLAND CLARKE PLACE, RESTON, VA, 20191, US

(73) Assignee: RIKEN, Saitama (JP)

(21) Appl. No.: 10/292,906

(22) Filed: Nov. 13, 2002

(30) Foreign Application Priority Data
Nov. 13, 2001 (JP) 347316/2001

(51) Int. Cl. C12N 5/00; C12N 5/08

(52) U.S. Cl. 435/325; 435/368; 435/289.1

(57) ABSTRACT

An object of the present invention is to provide an electrical stimulation apparatus for efficiently giving direct electrical stimulation to a large number of nerve cells ex vivo without causing any injuries to the cells. The present invention provides a cell stimulation apparatus for giving electrical stimulation to cultured cells in a non-contact state to the cultured cells, which comprises: a plurality of positive and negative electrodes which extends in an identical direction and are installed on a supporting plate; and supporting means which supports the plural positive and negative electrodes at a position having a predetermined distance from the cultured cells.

### TECHNICAL FIELD

The present invention relates to a cell stimulation apparatus and a cell stimulation method. More specifically, the present invention relates to a cell stimulation apparatus and a cell stimulation method wherein electrical stimulation can be given to cultured cells in a non-contact state to the cultured cells.

### BACKGROUND ART

One of the features of nerve cells is that they have an electrical activity which is referred to as nervous activity. It is thought that, as immature nerve cells differentiate and develop, the nerve cells express ion channels, transmitter receptors or the like in response to stimulation of neurotransmitters, neurotrophic factors or the like, and acquire sensitivities to the transmitters or excitability (inherent nervous activity), which are inherent to nerve cells, and their patterns express the history or individuality of each nerve cell. In recent years, it has been shown that this nervous activity, that is patterned electrical stimulation, inversely controls behaviors of substances associated with the nerve.

The present inventors have made studies in order to clarify nervous activity and the biological functions. As a result, it has been revealed that there is a possibility that a pattern of nervous activity (a pattern of electrical activity) works as an information code system. Although the ultimate goal of the present inventors' research is to control brain plasticity by controlling the pattern of nervous activity, important points for that goal are to clarify the role of nervous activity patterns (neuronal impulses), and to decode and profile these patterns of nervous activity. For this purpose, it has been necessary to conduct experiments under artificially well-controlled stimulation conditions.

Further, the control of electrical activity of nerve cells enables the control of brain plasticity, and is applicable for treatment. In the field of regenerative medicine, particularly nerve regeneration, it is considered to be essential to allow nerve cells to have electrical activity in order to acquire normal functions. To that end, an electrical stimulation apparatus, whereby direct electrical stimulation can efficiently be given to a large number of nerve cells in vitro without causing any injuries to the cells, would be necessary.

Known conventional methods for giving electrical stimulation to cells are: (1) a method wherein electrodes are inserted into nerve cells to stimulate the cells; and (2) a method wherein nerve cells are adhered to a stimulation electrode base to stimulate the nerve cells. However, according to method (1), it is difficult to stimulate a large number of cells at same time and also it has a drawback that it causes much damage to cells. Further, according to method (2), the number of nerve cells to be adhered to the interface of electrode base is very small and therefore it is difficult to make it work as a practical stimulation apparatus.

### SUMMARY OF THE INVENTION

The object of the present invention is to solve the above-mentioned problems of the background art. Namely, one object of the present invention is to provide an electrical stimulation apparatus for efficiently giving direct electrical stimulation to a large number of nerve cells ex vivo without causing any injuries to the cells. Another object of the present invention is to provide a cell stimulation method which can efficiently give direct electrical stimulation to a large number of nerve cells ex vivo without causing any injuries to the cells.

The present inventors made intensive studies in order to solve the above objects. As a result, they have found that desired electrical stimulation can be given to cells by using a cell stimulation apparatus which comprises a plurality of positive and negative electrodes which extend in an identical direction and are installed on a supporting plate, and supporting means which supports the plural positive and negative electrodes at a position having a predetermined distance from cultured cells, thereby accomplishing the present invention.

According to the present invention, there is provided a cell stimulation apparatus for giving electrical stimulation to cultured cells in a non-contact state to the cultured cells, which comprises: a plurality of positive and negative electrodes which extends in an identical direction and are installed on a supporting plate; and supporting means which supports the plural positive and negative electrodes at a position having a predetermined distance from the cultured cells.

According to preferable embodiments of the present invention, there are provided:

the cell stimulation apparatus which further comprises container supporting means which supports inside a base frame a culturing container for keeping the cultured cells, and a plurality of connectors connectable to the plural positive and negative electrodes which are provided inside the base frame, wherein the plural connectors are connected to an electrical signal generation device;

the cell stimulation apparatus wherein the base frame is provided with an adaptor connectable to the electrical signal generation device and each of the plural connectors is connected to the adaptor by wires inside the base frame;

the cell stimulation apparatus wherein the container supporting means is detachable from the base frame;

the cell stimulation apparatus wherein the plural positive and negative electrodes are arranged at intervals of 3 to 10 mm;

the cell stimulation apparatus wherein the plural electrodes are individually controlled; and

the cell stimulation apparatus wherein the distance between the cells and the electrodes is kept constant by measuring impedance.

According to another aspect of the present invention, there is provided a method for electrically stimulating cells, which comprises: setting a plurality of positive and negative electrodes in a culturing plate containing cultured cells, in an identical direction toward a face formed by the cultured cells so that the electrodes do not contact with the cells; forming an electric field using the electrodes; and stimulating the cultured cells by the electric field.

According to preferable embodiments of the present invention, there are provided:

the method for electrically stimulating cells wherein the cultured cells are nerve cells;

the method for electrically stimulating cells wherein the plural positive and negative electrodes are arranged at intervals of 3 to 10 mm;

the method for electrically stimulating cells wherein the cultured cells are stimulated by staggering the timing of electrical stimulation by means of the plural positive and negative electrodes; and

the method for electrically stimulating cells wherein the cells are electrically stimulated using the above-mentioned cell stimulation apparatus of the present invention.

### BRIEF DESCRIPTION OF THE DRAWINGS

FIG. 1 is a front view illustrating a base frame of a cell stimulation apparatus according to the present invention and members provided thereon.

FIG. 2(a) illustrates a front side of a changeover device and FIG. 2(b) illustrates a rear side thereof.

FIG. 3 is a view illustrating a connecting state between the changeover device and an isolator.

FIG. 4 is a side view illustrating a state wherein an electrode device is set up with a petri dish containing cells.

FIG. 5 is a partially cutaway top view illustrating a state wherein the electrode device is set up with the petri dish containing cells.

FIG. 6 is a top view illustrating a state wherein a plurality of petri dishes are arranged inside the base frame and electrical stimulation is given to cells.

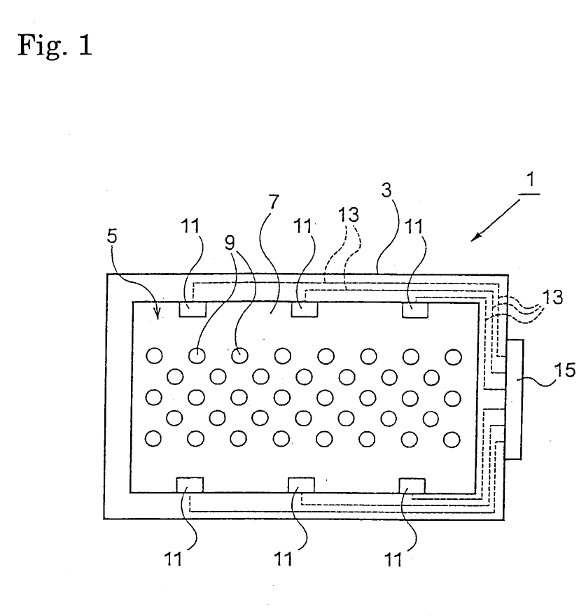

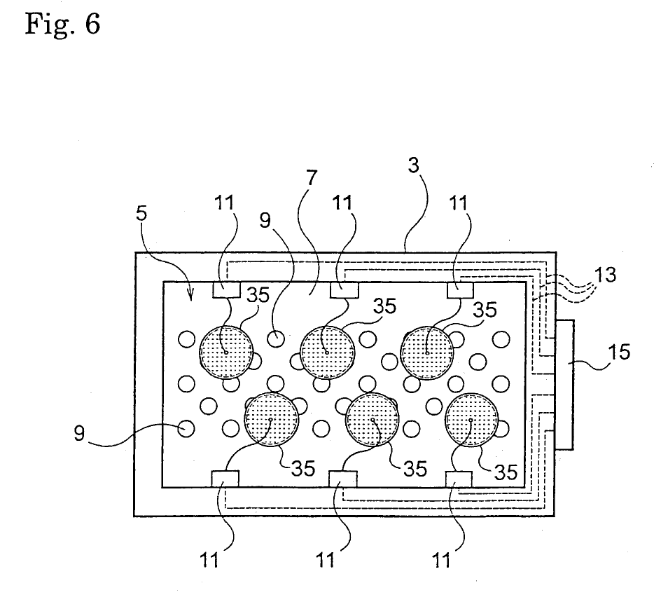

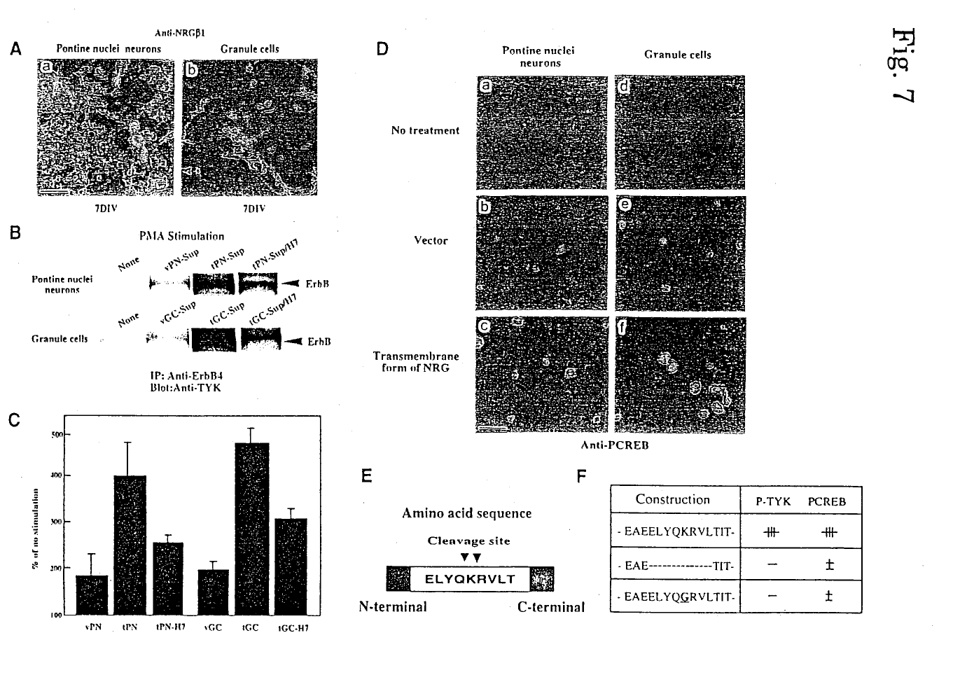

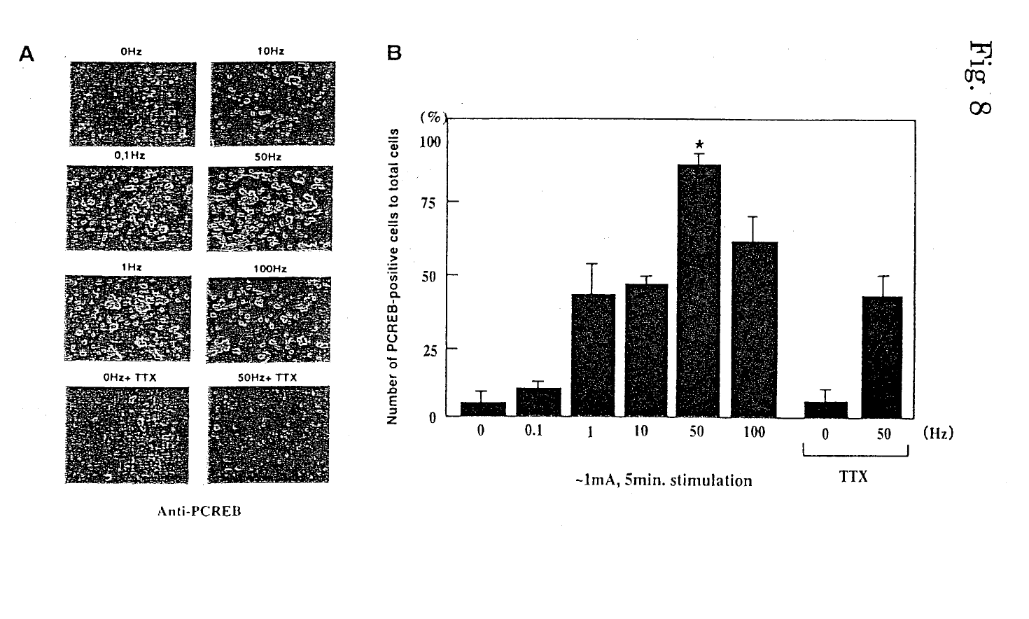

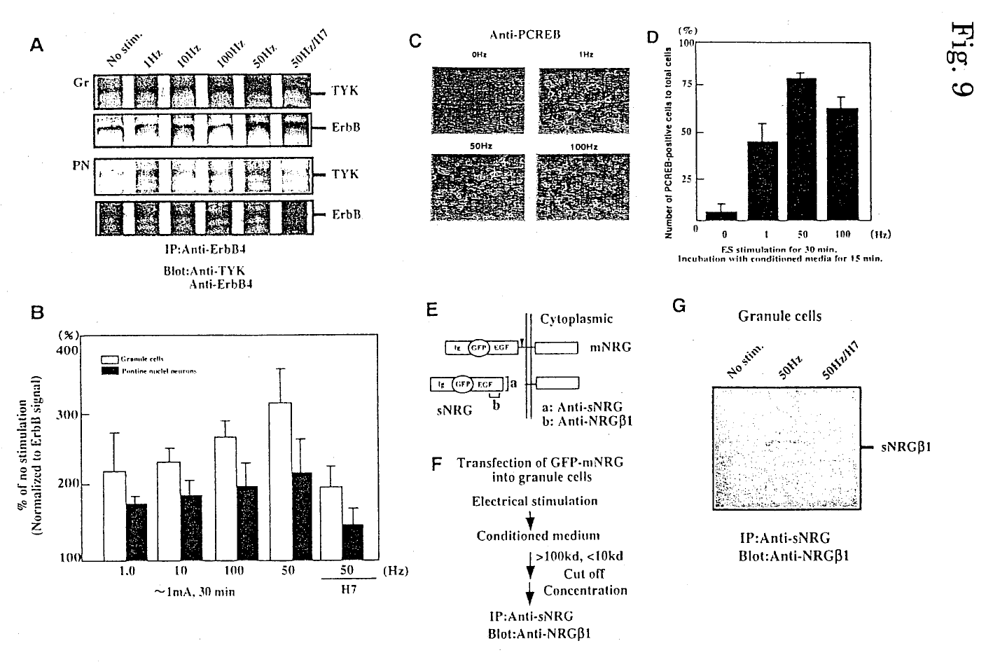

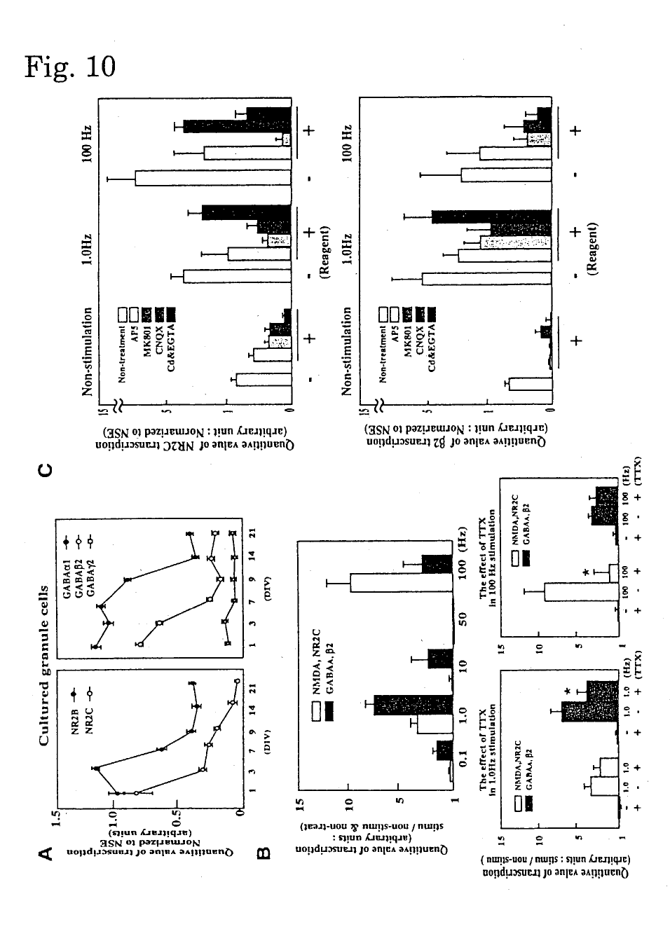

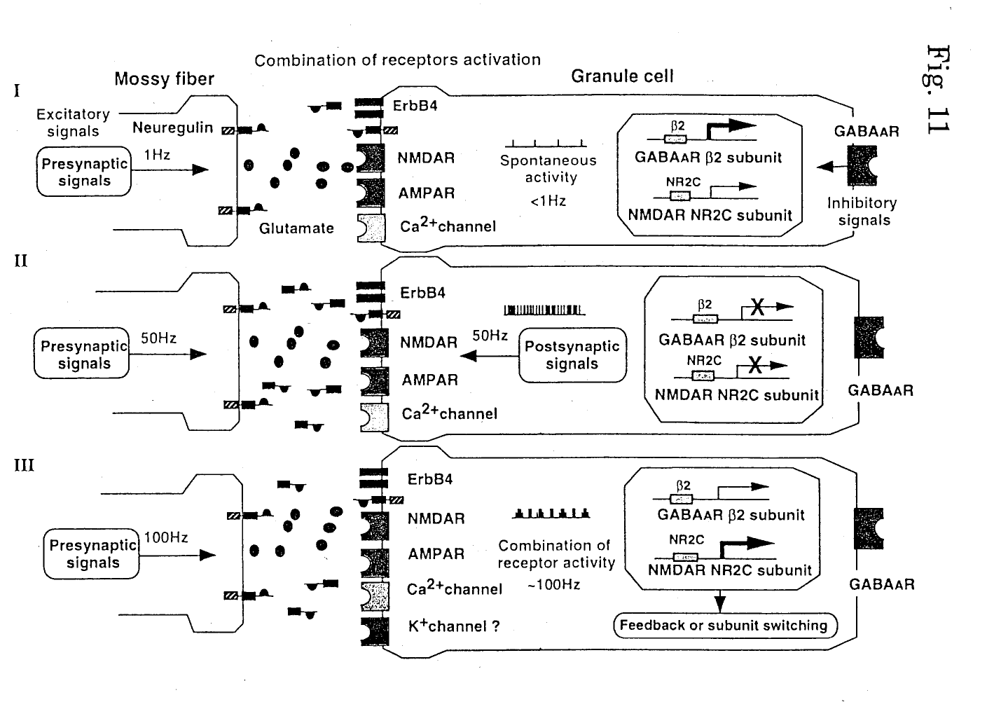

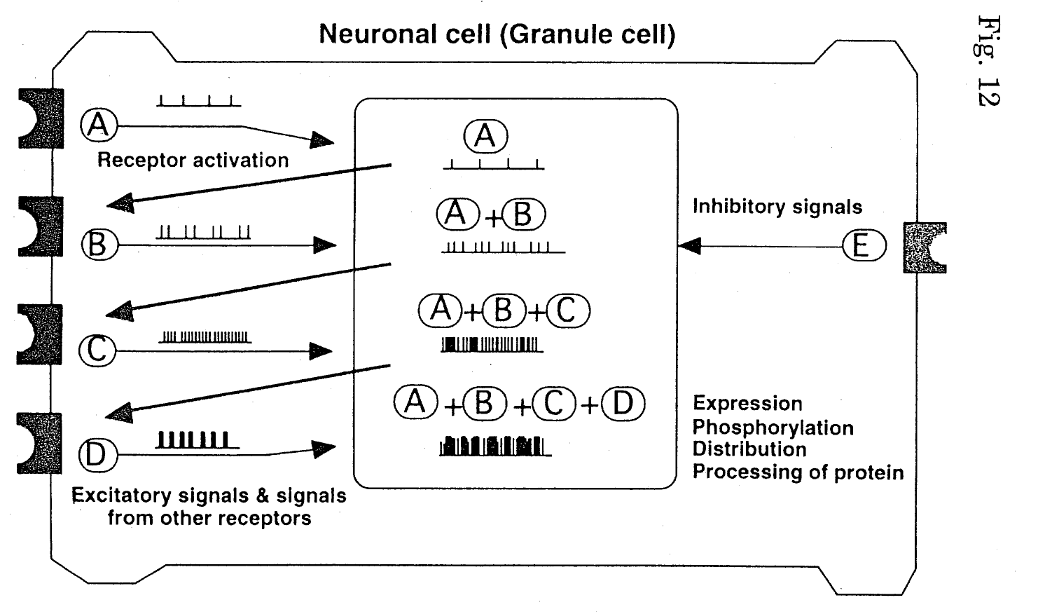

Each number in FIGS. 1 to 6 represent the following meanings:

1 cell stimulation apparatus

3 base frame

5 storage portion

7 culturing tray

9 hole

11 connector

12 connector at a base terminal side of electrode

13 wiring

15 adaptor

17 changeover device

19 isolator

21 connection terminal

23 switches for setting information of each channel

25 switches for setting pulse of each channel

27 ON-OFF switches

28 main power supply

29 output connection portion

31 connection outlet

33 input portion

35 electrode device

37 cell

39 petri dish

41 supporting plate

43 wiring

45, 45a, 45b electrode

47 lid portion

49 medium

FIG. 7 shows proteolytic cleavage of neuregulin by PMA stimulation.

Pontine nuclei neurons and cerebellar granule cells were stained with anti-NRG β1 antibody at 7DIV(days in vitro). In both types of neurons, the cell bodies and neuronal process were NRG positive (A). Scale bar; 60 μm.

In B, pontine nuclei neurons and granule cells with and without transfection of the transmembrane form of NRG were prepared and stimulated by PMA for 60 minutes and then the conditioned media were collected. Tyrosin-phosphorylation activity of the conditioned media was examined using cerebellar granule cells. The granule cell cultures were stimulated for 5 to 10 minutes with the conditioned media that were collected and concentrated. Lysates from granule cells were resolved by SDS-PAGE, and the blot was probed with an anti-phosphotyrosine antibody (4G10) after immunoprecipitation with anti-ErbB4 antibody. Tyrosine-phosphorylated bands of 180 kD were observed by stimulation (B). The conditioned media were collected from PN(non-transfected pontine neuclei neurons), tPN(transfected pontine neuclei neurons), GC(non-transfected granule cells), and tGC(transfected-granule cells). The result is summarized in C. The experiments were repeated independently three or four times.

In D, using cerebellar granule cells, CREB-phosphorylation was checked after the conditioned medium stimulation. Serum-starved cerebellar granule cells were treated (for 5 to 10 minutes) with conditioned media collected from pontine nuclus neuron and granule cell cultures. The conditioned media from pontine nuclei neurons was used for stimulation in a, b and c. In d, e and f, conditioned media were prepared from granule cell culture.

a, d: control,

b, e: vector (pEGFP-N3),

c, f: pNRG-GFP.

The stimulated granule cells were stained with a phospho-CREB antibody after fixation. The soluble forms released by PMA stimulation (60 minutes) were enriched and added into cultured granule cells. The conditioned media should contain endogenous NRG and recombinant NRG in panel c and f. The subtraction from b and e showed the effect of NRG cleaved from recombinant mNRG Using ErbB and CREB phosphorylation assay systems, amino acid sequences required for proteolytic cleavage were identified in E and F. ELYQKRVLT sequences were located on the extracelluar position at the N terminal side of the transmembrane domain. When the sequences was deleted or mutated from lysine to glysine, the efficiency of proteolytic cleavage was inhibited as shown in F. The wide area including lysine residue was essential for recognition by protease.

FIG. 8 shows CREB-phosphorylation activity by electrical stimulation.

Pontine nuclei neurons and cerebellar granule cells were prepared from 18-day-old embryonic mice and postnatal day 7 mice, respectively. The neurons were cultured for 7 days, and then stimulated with different patterns of electrical stimulation. After electrical stimulation, CREB-phosphorylation was examined using granule cells (A and B). The positive cells with anti-PCREB antibody were counted and normalized to the total cell numbers. Five random points of microscopic view (20×magnification) from each dish were photographed for cell counting. From independent experiments, 3 to 5 dishes were counted. The highest efficiency of CREB phosphorylation was observed at 50 Hz under the continuous 5 min stimulation. The phosphorylations were partially blocked by TTX.

FIG. 9 shows proteolytic cleavage of NRG by electrical stimulation.

After electrical stimulation, tyrosin-phosphrylation activity of ErbB4 was measured by the same methods with PMA stimulation. Panel A shows that tyrosin-phosphorylation activity of the conditioned media to ErbB4 had the highest efficiency at 50 Hz stimulation in the pontine nuclei neurons and granule cells. The tyrosine phosphorylation was blocked by PKC inhibitor, H7. The results are summarized in the graph (see B). In C and D, CREB-phosphorylation was checked after stimulation with the conditioned medium stimulation using cerebellar granule cells. Serum-starved cerebellar granule cells were tested (for 15 minutes) with conditioned media collected from granule cell cultures after electrical stimulation. At 50 Hz stimulation, 77.4±2.08% of granule cells was PCREB positive, and 62.5±4.17% were PCREB positive at 100 Hz stimulation. The reaction peak of PCREB was observed at 50 Hz (n=15, *P<0.015).

Finally, the cleaved NRG was directly detected by immunobloting after immunoprecipitation as shown in G. The detection strategy is summarized in E and F. The conditioned media were collected from 5×10⁷ to 5×10⁸ granule cells to which mNRG was transfected after electrical stimulation (transfection efficiency; ˜5%). The media were concentrated using centricon, and then immunoprecipitated with anti-sNRG antibody. Anti-sNRG polyclonal antibody (prepared in Example 1) that recognizes only the c-terminal of cleaved NRG was used as an antibody. Western blot analysis was performed with anti-NRGβ1 antibody that recognizes only the NRGβ1 form after immunoprecipitation. As shown in G, the signal of cleaved NRG could be detected at 50 Hz stimulation. The signal disappeared with PKC inhibitor H7. From these results, the amount of released NRG in the media appears to be different at different frequencies of stimulation.

FIG. 10 shows NMDA and GABAA receptor subunit expressions quantified by real-time quantitative RCR method.

In A, NMDA and GABAA receptor subunit expressions were examined using cultured granule cells with 10 mM KCl from 1 to 21 days in vitro (DIV). 7DIV was chosen for experiments of electrical stimulation. At 7DIV, granule cells are still sufficiently alive, however NMDA receptor, NR2C, 2B and GABAA receptor β2 subunits mRNA were decreased. Both GABAA receptor α1 and γ2 mRNA were retained at 7DIV. Real-time quantitative analyses of RT-PCR were performed after electrical stimulation of different patterns. The transcription of NR2C and β2 subunits was controlled by different frequencies. The NR2C transcription was promoted by 1.0 and 100 Hz stimulation (B), and the increase observed at 100 Hz stimulation was blocked by TTX treatment. However, the increase at 1.0 Hz was not strongly blocked by TTX. On the other hand, β2 transcription was promoted at 0.1 to 20 Hz stimulation more than that of NR2C, and the increase was blocked by TTX treatment. The increase in 100 Hz was partially blocked by TTX. 0.1 Hz; n=6, 1 Hz; n=18, 10 Hz; n=10, 50 Hz; n=12, 100 Hz; n=26. Non-stimulation with TTX; n=3, 1 Hz and TTX; n=6, 100 Hz and TTX n=6, *p<0.001, **p<0.00001.

Pharmacological experiments were carried out under electrical stimulation in C. In the case of NR2C, the elevated transcription at 1 and 100 Hz was partially blocked by all antagonists and blocker. However, particularly the MK801 strongly blocked NR2C mRNA expression strongly. In the case of β2, CNQX in addition to MK801 blocked transcription at 1.0 and 100 Hz stimulation. The mRNA elevation at 100 Hz was inhibited strongly by non-specific calcium blocker, however the increase at 1.0 Hz was not obviously blocked. In all cases, direct electrical stimulation could partially minic receptor activation at least until the basal level. AP5; competitive NMDA receptor antagonist, MK801; non-competitive NMDA receptor antagonist, CNQX; AMPA receptor antagonist, Cd & EGTA; non-specific calcium channel blocker. Each of the experiments were independently repeated from 3˜8 times.

FIG. 11 shows schematic diagram of NMDA and GABAA receptor subunit expression controlled in a frequency-dependent manner.

Cerebellar granule cells receive excitatory signals from mossy fibers, and inhibitory signals from Golgi cells via GABAA receptors. A combination of these signals decides the patterns of neuronal activity. Even granule cells that are not innervated by mossy fibers have spontaneous activity. Under a condition of relatively low frequency, both NR2C and β2 subunit expressions were observed, but the β2 expression was promoted more than NR2C (I). On the other hand, NR2C expression was induced more at high frequency such as 100 Hz. NR2C expression may be induced in mossy fiber innervating granule cells, and that are involved in a considerable amount of receptor activation (III).

FIG. 12 shows model in receptor activation and patterns of neuronal activity.

### EMBODIMENTS OF THE INVENTION

Hereinafter, an embodiment of the present invention will be described based on the figures. FIG. 1 is a front view illustrating a base frame 3 of a cell stimulation apparatus 1 according to the present invention, and members provided thereon. The base frame 3 is formed in a rectangular shape using a hollow member, and a storage portion 5 is formed inside the rectangular shape. Further, on the storage portion 5, there is installed a culturing tray 7, in a upwardly detachable manner, which is container supporting means for placing a petri dish (not shown in FIG. 1) that is a culturing container containing cells. Over the length and breadth of the culturing tray 7, several holes 9 are formed, through which gas or steam may pass.

Inside the base frame 3, for example, six connectors 11 are provided and these connectors 11 are each connectable to a connector at a base terminal side of an electrode device, which will be described below. Wires 13 extend from respective connectors 11. Wires 13 go through the hollow member constituting the base frame 3, and the opposite ends thereof are each connected to an adaptor 15 provided outside the base frame 3.

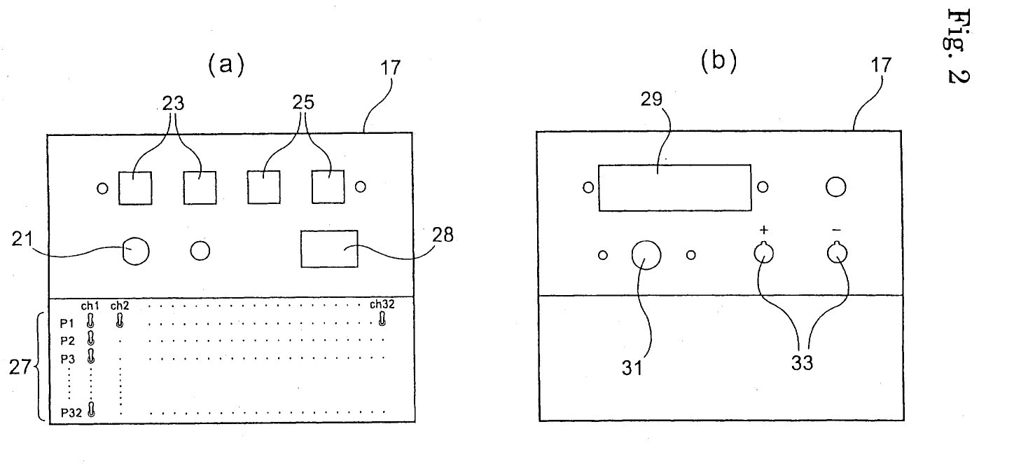

The adaptor 15 is connectable to a changeover device 17 shown in FIGS. 2(a) and 2(b) via suitable wiring. FIG. 2(a) shows a front side of the changeover device 17 and FIG. 2(b) shows a rear side of the changeover device 17. The changeover device 17 of this embodiment mounts thereon a transistor which allows pulse current to an electrode to be changed over at 1 mm per second in the shortest length.

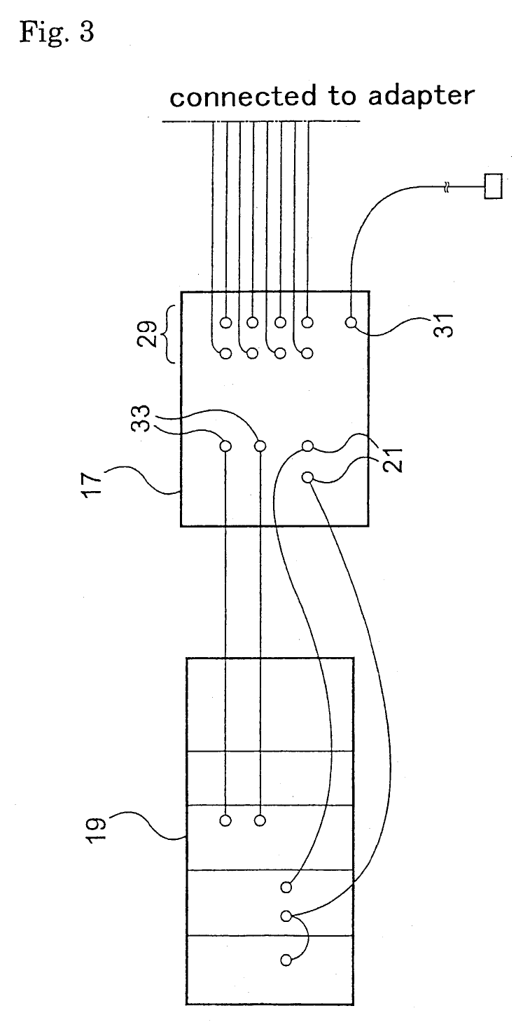

As shown in FIG. 2(a), on the front side of the changeover device 17, there are provided: a connection terminal 21 for establishing connection to an isolator 19 (see FIG. 3); switches 23 for setting the information of each channel; switches 25 for setting pulse of each channel; ON-OFF switches 27 for each channel; and a main power supply 28. The ON-OFF switches 27 are composed of a group of switches arranged in a matrix comprising ch1, ch2, . . . ch32 in a horizontal direction and p1, p2, . . . p32 in a vertical direction, by which a position is specified. Among these, any two or more switches are selected and turned on, and thereby pulse current can selectively be supplied between the respective electrodes, which will be described below.

Further, as shown in FIG. 2(b), on the rear face side of the changeover device 17, there are provided: an output connection portion 29 to the adaptor 15; a connection outlet 31 connectable to a 100 V power source; and an input portion 33 which receives a signal from the isolator 19.

FIG. 3 is a view illustrating a connecting condition between the changeover device 17 and the isolator 19. The isolator 19 is a device which controls electricity so as to enable precise electrical flows to electrode devices 35 which will be described below. When pulse is simultaneously supplied to the plurality of electrode devices 35, it is necessary to prepare the corresponding number of isolators 19 to the electrode devices 35. On the other hand, when the timing of pulse supply is staggered toward each of the plural electrode devices 35, one isolator 19 is sufficient for the pulse supply. This point will be explained below.

As shown in FIG. 3, electricity which is sent from the connection terminal 21 of the changeover device 17 to the isolator 19 is converted at the isolator 19 to electric signals with pulse number corresponding to a predetermined electrode, and the pulse signals are inputted into the input portion 33 of the changeover device 17. The pulse signals that have been inputted into the input portion 33 are supplied from the output connection portion 29 via the adaptor 15 and then from a prescribed connector 11 to the electrode device described below. In addition, the changeover device 17 and isolator 19 are designed for the electric signal generation device.

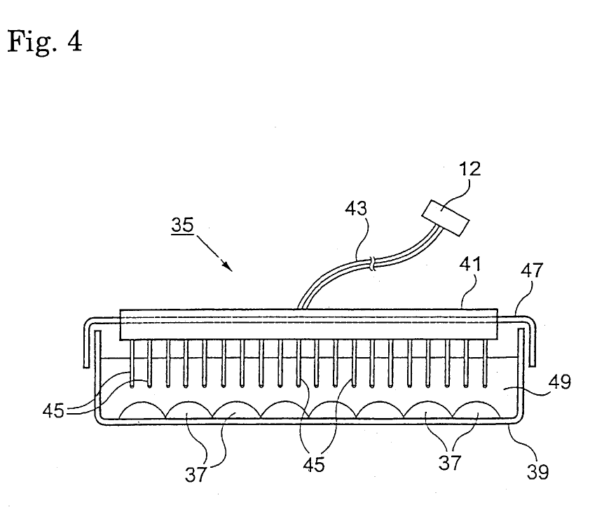

Hereinafter, electrode devices 35 having a characteristic configuration of the present invention will be described by referring to FIGS. 4 to 6. FIG. 4 is a side view illustrating a condition wherein one electrode device 35 is set up with a petri dish 39 containing cells 37. The electrode device 35 is provided with a supporting plate 41, and a wiring 43 is extended from a top surface of the supporting plate 41. The wiring 43 has the base terminal side connector 12 formed on an end thereof, which is connectable to the connector 11. In addition, a plurality of positive and negative electrodes 45 arranged at intervals of 3 to 10 mm are downwardly extended from the under surface of the electrode device 35.

The electrode device 35 is supported in an opening formed on a lid portion 47 of the petri dish 39, and the electrodes 45 are downwardly extended from the electrode device 35. Although at least a tip portion of each electrode 45 is inserted into medium 49 in the petri dish 39, the tip portion is kept at such a height that it does not have direct contact with the cells 37. The structure for keeping the electrodes 45 at such a height is not limited to those shown in FIG. 4. For example, the structure can be realized by supporting the electrode device 35 itself directly on an upper end of the petri dish 39 or supporting the electrode device 35 by use of a suitable supplemental tool so that the tips of electrodes 45 do not have direct contact with the cells 37.

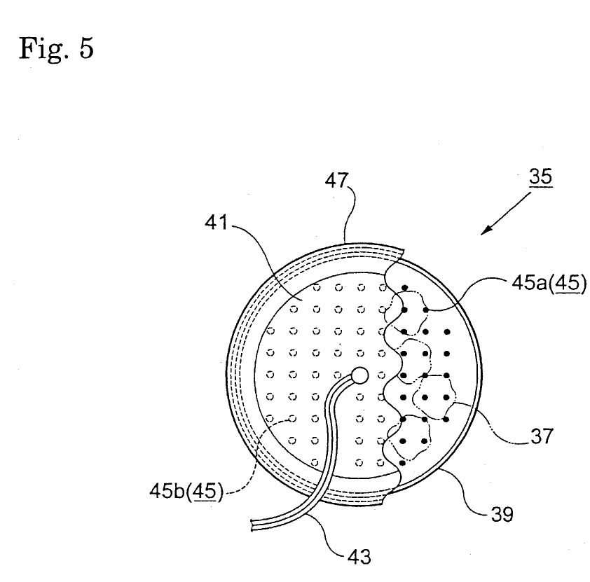

Portions other than the tips of electrodes 45 are coated with insulating varnish and thus current is applied into the medium 49 only through the tip portions of electrodes 45. The electrodes 45 are arrayed equidistantly and orderly as shown in FIG. 5. Among the above-mentioned ON-OFF switch 27, for example, two switches are selectively turned on, thereby carrying a predetermined pulse current between two electrodes 45a and 45b corresponding to the turned-on switches.

When one isolator 19 is used for supplying pulse current to a plurality of electrodes 45, pulse current is supplied to a first set of electrodes, and after a fixed interval another pulse current is supplied to a second set of electrodes. Thereafter, another pulse current is supplied to a third to n-th sets of electrodes at the fixed intervals. The process of repeating this operation can be adopted.

There are supposed to be 10 electrodes 45 that make five sets of positive and negative electrodes. For example, when it is desired to give 1 Hz stimulation through all the sets for 30 minutes, one stimulation per one second is supplied to the first set for 30 minutes. Stimulation through the second set is given 1.1 milliseconds later after the pulse current through the first set is flowed, and pulse current through the third, fourth, and fifth sets is flowed at 1.2, 1.3, and 1.4 milliseconds later, respectively. Thereafter, pulse current is again flowed through the first set. This cycle is repeated. After 30 minutes, this allows stimulation to be evenly given to the entire cell culture within an error of 30 milliseconds. According to a condition on how the stimulation as a whole is designated, stimulation timing through each set can be changed. According to the above embodiment, different timings to supply pulse current are applied to all the five sets of electrodes. However, by using two isolators 19, for example, one and the other stimulations are given from the first and third sets of electrodes at the same time, thereafter the following one stimulation is given from the second, the third, . . . sets and the following other stimulation is given from the fourth, fifth, ... sets. In this manner, use of two circulation patterns to orderly give stimulation from the electrodes is applicable.

Hereinafter, the present invention will be described by referring to Examples, but the present invention is not limited by the Examples.

### EXAMPLES

### Example 1

Preparation of antibodies which specifically recognize secretory neuregulin

(1) Design of Antigen Hapten Peptides

In order to prepare anti-peptide antibodies which complements limited proteolytic reaction, information concerning the cleavage site of a target substrate protein is necessary. In this example, a peptide wherein a cysteine residue is added to a short peptide (5 mer or 6 mer) containing C-terminal of secretory neuregulin, was synthesized for use as a hapten. To be more precise, a mixed peptide of Cys-Glu-Leu-Tyr-Gln and Cys-Glu-Leu-Tyr-Gln-Lys was used as antigen.

(2) Used Reagents

synthesized hapten peptide

KLH(keyhole limpet hemocyanin) in 50% glycerol (about 80% mg/ml) (Calbiochem)

DMFA (dimethylformamide)

MBS (m-maleimidobenzoyl-N-hydroxysuccinimide ester) (Pierce)

gel filtration column (Pharmacia PD-10)

50 mM sodium phosphate buffer (pH 7.5)

100 mM sodium phosphate buffer (pH 7.2)

Immunity

Freund's complete adjuvant (FCA)

Freund's incomplete adjuvant (FIA)

syringe, injection needle, etc.

Affinity Purification of Antibodies

100 mM HEPES buffer (pH 7.5)

Affigel 10 or 15 (BioRad)

30% acetic acid

20% ethanol

PBS

50 mM citrate buffer (pH 3.0)

2M TRIS buffer (pH 9.5)

20 % glycerol-containing Na-PBS

(3) Preparation of Antigens (Hapten/Carrier Complexes)

(a) MBS/activated KLH is prepared. Approximately 40 mg (0.5 ml) of KLH was added to 1.5 ml of 50 mM sodium phosphate buffer (pH 7.5), and the mixture was stirred by a stirrer. Then, a solution prepared by dissolving 9.3 mg of MBS into 0.38 ml of DMFA (to be prepared before use) was added thereto. After addition of MBS, the obtained solution was stirred at room temperature for 30 minutes. Thereafter, it was centrifuged at 2,000 rpm for about 2 minutes, and the supernatant was used below.

(b) MBS/activated KLH is removed from free MBS. Pharmacia PD-10 column was washed with 40 to 50 ml of 50 mM sodium phosphate buffer (pH 7.5) for equilibration. 2 ml of the supernatant obtained by centrifugation in process (a) was added to the column and infiltrated with a gel, and then 0.5 ml of the buffer was added thereto. After the infiltration was completed, eluate collection was started (first 2.5 ml of the eluate was discarded as prevoid), and 2 ml of the eluate (MBS/activated KLH) was collected. By this operation, standard sample which can be used for 4 couplings can be obtained.

(c) Synthesized hapten peptide is coupled with activated KLH. Approximately 5 mg of synthesized peptide was dissolved into 4.5 ml of 100 mM sodium phosphate buffer (pH 7.2) and stirred. While dissolving, pH test paper was used to confirm that the pH of the solution was not reduced. 0.5 ml of MBS/activated KLH was added thereto, and the mixture was stirred at 4° C. all day long. It is not necessary to carry out dialysis thereafter. The obtained products were used as antigens. They were preserved at −20° C. or −80° C.

(4) Immunization

(a) Polyclonal antibodies were prepared using rabbits. Initially, a primary immunization was given to rabbits with individual weights of approximately 3 kg. 0.6 ml of FCA was added to 0.3 ml of antigen solution, and after stirring, emulsion thereof was prepared by sonication [Branson Sonifier 185 (bath type), power 7 to 10, approximately 3 minutes]. The obtained emulsion was divided and injected into the rabbits at about 10 subcutaneous portions of both right and left back muscles. 18G or 21G injection needles were used.

(b) A second immunization was performed after approximately 1 month. In this case, emulsion was prepared in the same manner by adding 0.6 ml of FIA to 0.3 ml of the antigen solution. The obtained emulsion was injected into the right and left thigh muscles.

(c) 2 weeks and 4 weeks after the second immunization, third and fourth immunizations were performed. In this case, 0.15 ml of antigen solution was diluted with 0.45 ml of PBS, and subcutaneous injection was carried out into the back in the same manner as (a). 26G injection needles were used.

(d) Approximately 1 week after the fourth immunization, partial blood collections were conducted. Approximately 40 to 50 ml of blood was collected and checked for antibody production, and then affinity purification was conducted. When the results were favorable, 2 additional immunizations were performed after an approximately one-month rest, and then collections of whole blood were conducted.

(5) Affinity Purification of Antibodies

(a) Affinity gel is prepared. As an affinity carrier, Affigel 10 or 15 was used. First, 1 to 5 mg of hapten peptide was dissolved in 4 ml of 100 mM HEPES buffer (pH 7.5). Next, 1 to 2 ml of Affigel was washed on a glass filter by aspiration (twice with 10 ml of ice-cold distilled water), and then immediately added to the peptide solution. After rotating and stirring the obtained solution all day long at 4° C., aspiration washing was again conducted on a glass filter to remove free peptides. For this case, the obtained product was completely washed with 30 % acetic acid or 20 % ethanol in addition to a sufficient amount of distilled water, and was finally equilibrated with PBS. The obtained product was preserved in a cold storage.

(b) Specific antibodies are adsorbed onto an affinity column. Affinity gel was packed into a column (with an internal diameter of approximately 5 to 10 mm), and washed with PBS. 10 ml of inactivated serum was diluted with an equal amount of PBS and passed through a filter (0.22 or 0.45 μm), and then added to the column. Permeated liquid was collected, and the addition of the permeated liquid was repeated 3 to 4 times (at a flow rate of approximately 1 ml/minute). Further, the column was washed with approximately 50 ml of PBS.

(c) Antibodies are collected. 0.5 ml of 2 M TRIS buffer (pH 9.5) was first poured into a tube, and antibodies eluted from the affinity gel were collected into the tube. The elution was conducted by adding 5 ml of 50 mM citric acid buffer (pH 3.0) at a flow rate of approximately 1 ml/minute. Next, the eluate was transferred to a dialysis tube, and dialyzed against Na-PBS containing 20% glycerol (all day long at 4° C.). The quantitative determination of the antibodies was carried out by measuring absorption at 280 nm (1 mg IgG/ml, A₂₈₀=1.4). Usually, 1 to 10 mg of specific IgG is collected. After dispensing, the obtained antibodies were preserved at −80° C.

### Example 2

(Method)

(1) Cell Preparation

Cerebellar granule cells and neurons of the pontine nuclei were prepared by the standard method from P7 and E18 BALB/c mice, respectively. 7 DIV cultures were used for PMA and electrical stimulation in both types of cells. The granule cells were cultured for 121 days in vitro (DIV) under the 10 mM KCl condition for quantitation of receptor subunits expression. The pontine nuclei neurons were cultured with DMEM (Gibco BRL) including 10% horse serum for 1 or 2 days, after that maintained with Neurobasal medium (Gibco BRL) supplemented with B-27 (Gibco BRL). The cultures were fed with a medium consisting of Neurobasal medium (Gibco BRL) supplemented with B-27 (Gibro BRL). The full-length of the NRG plasmid including the GFP-tag and vector (pEGFP, Clonthech) were transfected by Lipofectamine™ 2000 (Gibco BRL) respectively (transfection efficiency of pontine nuclei neurons; 1˜3%, granule cells; 5˜10%). At 24 to 36 hours after transfection, the neurons were stimulated with 1 82 M PMA (Tocris) for 60 minutes.

(2) Detection of Cleaved Form of NRG

The electrical stimulation in this Example was carried out using a cell stimulation apparatus having a structure as mentioned hereinabove and shown in FIGS. 1 to 6.

After electrical stimulation (1 mA, 30˜60V outside the cells) for 30 minutes on 5×10⁷18 5×10⁸ cells which were transfected with recombinant full length mNRG, the conditioned media obtained from the cells were collected and concentrated using centricon 10 and 100 (Millipore) to cut off large (>100 kd) and small (<10 kd) molecular weight proteins. The granule cells of 7DIV were treated with the concentrated conditioned media obtained from the pontine nuclei neurons and the granule cells. In the ErbB phosphorylation, Western blot analysis was performed by the standard method using a mouse monoclonal anti-phosphotyrosine antibody (4G10) after immunoprecipitation with a polyclonal anti-ErbB4 antibody (Santa Cruz) (Rieff H I, et al., J Neurosci, 19(24), 10757-10766 (1999). For immunoprecipitation studies, lysates were inclubated with the appropriate dilution of immunoprecipitating antibody for 1 hour at 4° C., followed by another 1-hour incubation at 4° C. with proteinA-Sepharose. Laysate were then centrifuged for 3 minutes at 15000 rpm, and the supernatant was discared. The pellets were then washed twice in lysis buffer and were resuspended in gel loading buffer. Samples were boiled for 3 minutes, and proteins were separated by electrophoresis.

To detect CREB phosphorylation, 7DIV cultured granule cells were fixed with 4% paraformaldehyde for 10 minutes after conditioned media stimulation for 10 to 15 minutes, and stained with a polyclonal anti-NRG β1 antibody, a polyclonal anti-PCREB antibody (BioLabs). The stained granule cells were observed with a laser confocal microscope (Carl Zeiss). Alexa™ dyes (Molecular probe) were used as a secondary antibody.

(Results)

(1) Proteolytic Cleavage of the Transmembrane Form of NRG in Pontine Nuclei Neurons and Cerebellar Granule Cells

The condition of dissociated primary cultures of cerebellar granule cells and pontine nuclei neurons used for the experiments in FIG. 7A was investigated. Pontine nuclei neurons (PN) were prepared from embryonic day 18 (E18) and cerebellar granule cells (GC) from postnatal day 7 (P7). To examine that proteolytic cleavage of mNRG occurs in cultured neurons, immunoprecipitation with anti-ErbB and anti-phosphotyrosine antibodies, and immunocytochemical analyses using anti-PCREB antibody were performed in pontine nuclei neurons and granule cells which were transfected with or without recombinant full-length NRG β1 including GFP-tag and then stimulated with the PKC activator, phorbol-12-myristate-13-acetate (PMA), for 60 minutes.

With regard to the NRG receptor in the synapses between mossy fibers and granule cells, it is previously reported that ErbB2 and ErbB4 are involved in the cerebellar system (Ozaki M, et al., Nature, 390, 691-694 (1997); and Ozaki M, et al., Neurosci Res, 30 (4), 351-354 (1998)). The expression of ErbB4 was more strongly observed in cerebellar granule cells in vitro and in vivo than ErbB2. Cyclic AMP responsive element binding protein (CREB) phosphorylation was involved further down stream of the ErbB4 signal transduction pathway (Taberbero A, et al., Mol Cell Neursci, 10, 309-322 (1998)). Conditioned media of the pontine nuclei neuron and granule cell cultures after treatment with PMA were collected, concentrated and applied into granule cells for 5 to 10 minutes. The lysates from granule cells were resolved by SDS-PAGE after immunoprecipitation with anti-ErbB4 antibody, and the blot was probed with anti-phosphotyrosine antibody (anti-TYK). Conditioned media was collected from non-transfected pontine nuclei neurons and granule cells (None), vector-transfected pontine nuclei (vPN), NRG-transfected pontine nuclei neurons (tPN), vector-transfected granule cells (vGC) and mNRG-transfected granule cells (tGC). Both the pontine nuclei neurons and granule cells which the NRG was transfected into, showed strong phosphorylation activity compared with non-transfected neurons and vector-only-transfected cells (see FIG. 7B, 7C). The amount of increased sNRG was checked by ErbB phosphorylation using granule cells. Tyrosine-phosphorylated bands of 180 kD with conditioned media obtained from mNRG-transfected neurons were clearly observed using PMA stimulation (FIG. 7B). The phosphorylation activity was suppressed by the conditioned media when the transfected pontine nuclei neurons and granule cells were treated with PKC inhibitor H7. Endogenous NRG did not show any obvious activity of ErbB phosphorylation. However when recombinant mNRG was transfected into the cultured neurons, ErbB4 phosphorylation was clearly observed. These results indicate that the sNRG was produced from recombinant mNRG following PKC activation. In FIG. 7C, the ratio of phosphorylation was normalized against ErbB4 signals that were blotted after immunoprecipitation with anti-ErbB4 antibody.

The result of CREB-phosphorylation is shown in FIG. 7D. The soluble forms released by PMA stimulation (60 minutes) were enriched using a spin column and added into the cultured granule cells. The stimulated granule cells were stained with anti-phospho-CREB antibody after fixation. The conditioned media from the pontine nuclei neurons was used for stimulation (a, b and c) and that from granule cell cultures for stimulation (d, e and f). Panel D shows controls (a, d), vector (b, e), and full-length NRG (c, f). The conditioned media (c and f) should have contained cleaved endogenous and recombinant NRG The subtraction of b from c and e from f indicated CREB-phosphorylation induced by sNRG that was from recombinant mNRG. A distinct CREB-phosphorylation was observed after 5 or more minutes of treatment with the conditioned media. An obvious release of NRG by KC1 stimulation could not be observed when living cultured granule cells were used.

The conditioned media obtained from pontine nuclei neurons and granule cells transfected with full-length mNRG showed distinct activity of ErbB- and CREB-phosphorylation. From measurement of ErbB- and CREB-phosphorylation activities, amino acid sequences necessary for proteolytic cleavage were identified. As shown in FIGS. 7E and 7F, deletion mutants inside the ELYQKRVLT are did not provide a clear proteolytic cleavage. Point mutation from K to G within this area also caused diminishment of cleavage as shown in the table, and FIG. 7F. Recently, NRG was reported as a substrate of the metalloprotease (ADAMs) family protease (Shirakabe K, et al., J Biol Chem, 276 (12), 9352-9358 (2000)). NRG cleavage by metalloprotease has been reported to be mainly in the Golgi-apparatus. A type of proteolytic cleavage of mNRG has already been reported to occur on the cell surface (Loeb J A, et al., Mol Cell Neurosci, 11 (1-2), 77-91 (1998)). The proteolytic cleavage of NRG may be regulated by several proteases, depending on the kinds of cells, the protein localization of NRG and protease, and timing.

(2) Proteolytic Cleavage of NRG by Patterned Electrical Stimulation

CREB-phosphorylation activity was measured by immunocytochemistry with anti-PCREB antibody to reveal the optimum point in CREB phosphorylation of cerebellar granule cells by electrical stimulation. The granule cells were directly electrically stimulated with different frequencies for 5 minutes (see FIG. 8A, 8B). The phosphorylation activity was observed with frequencies from 1 Hz to 100 Hz, and the optimum efficiency was found to be 50 Hz. The CREB-phosphorylation activity at 50 Hz was blocked 36.6±5.45% with the sodium channel blocker, TIX. In FIG. 8B, the PCREB-positive cells were counted and normalized against total cell numbers. These experiments suggest that the different frequencies produce different condition inside neuronal cells. 50 Hz was the most promising value to cause optimal proteolytic cleavage of NRG.

In order to ascertain if the proteolytic cleavage of NRG occurs at different patterns of electrical stimulation, ErbB phosphorylation was detected by immunoprecipitation with anti-TYK and anti-ErbB4 antibodies after electrical stimulation at different frequencies. The cleaved form of NRG pulled down with anti-ErbB antibody was detected by immunobloting with anti-TYK antibody. Anti-TYK was used to recognize phosphorylated ErbB receptors. The efficiency of phosphorylation was measured by nomalizing against ErbB4 signals. The phosphorylation signal was significantly stronger at 50 Hz stimulation than at any other frequency of stimulation (FIG. 9A, 9B). CREB-phosphorylation activity of the conditioned media also showed an optimal value at 50 Hz stimulation. The graphs in FIG. 8B and FIG. 9D show similar patterns.

After using the above methods, the cleaved form of NRG was detected directly by using the procedure described in FIGS. 9E, 9F. An antibody (anti-sNRG antibody) that recognizes only the c-terminal of the cleaved form of neuregulin was used. Approximately 1×10⁶ of transfected granule cells were used to collect conditioned media after electrical stimulation. Proteins of more than 100 kD and less than 10 kD were removed using centricon 10 and 100 centrifuge filtration, and were further concentrated by centricon 10. After that, immunoprecipitation was performed with anti-sNRG antibody, and immunoblotted with anti-NRG β1 antibody that could recognize only the β1 isoform of NRG. The blot is shown in FIG. 9G. The signal of the cleaved NRG was observed at a position of approximately 30 kD. In the case of 50 Hz stimulation with H7, an obvious signal of the cleaved form of NRG was not detected. From the data described here, it was concluded that the sNRG was produced from mNRG via proteolytic cleavage initiated and controlled by the specific pattern of electrical stimulation.

### Example 3

Analysis of Expression Mechanism of Transmission Receptor

(Method) Real-Time Quantitative Analysis of NMDA and GABAA Receptors Subunits

After electrical stimulation, a real-time quantitative analysis (ABI prism7700, Perkin Elmer) was performed. Primers and TaqMan probes were designed using primer Express (PE Biosystems). The PCR products amplified by each of the primers were a single band on agarose gel. The products were checked by direct sequence. Every primer which was used was not crossed to other genes. For the pharmacological experiments TTX (1 μM, Tocris), D-AP5 (50 μM, Tocris), MK801 (25 μM, Tocris), CNQX (10 μM, Tocris), Cd (100 μM, Wako Inc.) and EGTA (1 mM, Sigma) were used.

(Results) The Xxpression of NMDA and GABAA Receptor Subunits Controlled by Electrical Stimulation

The patterns of electrical activity which could control NMDA and GABAA receptor subunit expressions were examined. Using a real-time quantitative polymerase chain reaction (PCR) method, the mRNA expression of the NMDA and GABAA receptor subunits was quantified during culture from 1 through to 21 days in vitro. First, the mRNA expression level of each subunit of the NMDA and GABAA receptors in cultured granule cells was checked (FIG. 10A). Neural specific enolase (NSE) was used as a control. The properties of 7DIV (days in vitro) cultured neurons prepared from P7 mice are theoretically similar to those of P14 mice in vivo in maturation stage. At P14 in vivo, the NR2B expression is shut down in cerebellar granule cells, whereas NR2C expression is observed in all granule cells. The subunit switching of the NMDA receptors almost finished at P14 with the mice. On the other hand, α1, β2, and γ2 subunits of GABAA are abundantly expressed at P14 in vivo. To adjust in vitro conditions to match in vivo, granule cells that were cultured for 7 days in vitro (DIV) were chosen for electrical stimulation.

All of the cultured granule cells were stimulated at various frequencies (0˜100 Hz), mA for 30 minutes (FIGS. 10B, 10C). Although the cell bioavailability after stimulation was checked by chemical staining and anti-NRG antibody staining, a significant difference in the number of living cells was not observable in all cases. In the case of the NMDA receptor NR2B and GABAA α2, γ1, an obvious effect of electrical stimulation was not observed at any frequency. The expression of the NMDA receptor NR2C subunit was promoted by direct stimulation at frequencies of 1 and 100 Hz, and increases observed at high frequency stimulation such as 100 Hz were blocked by TTX. The NR2C elevation by stimulation at 1 Hz was not strongly blocked by TTX. In the case of the GABAA receptor β2 subunit, the mRNA expression increased with stimulation at low frequency, such as 0.1 to 10 Hz. The increase with low frequency stimulation was partially blocked by TTX, however the β2-increase with 100 Hz was not blocked by TTX.

Pharmacological experiments showed that the activity of NMDA and AMPA receptors, and calcium channels, were involved in retaining NR2C and β2 expressions. In NR2C at 1 Hz stimulation, the mRNA expression was strongly inhibited by NMDA, AMPA receptor antagonists. At a stimulation frequency of 100 Hz, in particular MK801 (a non-competitive NMDA receptor antagonist) strongly blocked NR2C elevation. In addition, calcium channels contributed more to the NR2C expression than to the AMPA receptor. In β2 at 1 Hz stimulation, the result was similar to that of NR2C at a stimulation frequency of 1 Hz. The mRNA increase of β2 at 100 Hz stimulation was inhibited by NMDA, AMPA receptor antagonists and calcium channel blocker (non-specific blocker; Cd & EGTA). In both cases of NR2C and β2, the calcium channel blocker strongly inhibited subunit expressions at high frequency, but did not inhibit at low frequency. It is understood that different frequencies control the combination of the involved granule cell receptors and the degree of activity. Moreover, specific electrical stimulation could partially restore the normal activity even with continued presence of antagonists and blockers. An example of this with the calcium channel blocker at 1 Hz stimulation is shown (FIG. 10C, in the case of β2).

(Discussion of Examples)

As shown in FIG. 11, cerebellar granule cells are thought to take a balance of excitatory and inhibitory signal input from mossy fibers and Golgi cells via NMDA and GABAA receptors. Patterns of neuronal impulses in granule cells will change during synaptic development by involvement of various receptors. The final intracellular electrical condition in granule cells is most likely determined by a combination of molecules such as the transmitter, neuropeptide, and neurotrophic factors as well as others from environment cues including presynaptic neurons. The different combination of molecules should make different patterns of neuronal impulses in the relationship between behavior of molecules and patterns of electrical activity. Some gene expressions have been reported to be regulated by patterned electrical activity (Buonanno A et al., Curr Opin Neurobiol, 9, 110-120 (1999)). It is certain that phosphorylation activity of molecules is controlled by patterned electrical activity (Buonanno A et al., Curr Opin Neurobiol, 9, 110-120 (1999)).

In Example 2, it is proved that protein processing such as proteolytic cleavage is controlled by patterned electrical activity. The proteolytic cleavage of NRG was observed from low frequency to high frequency, however the optimal proteolytic cleavage of NRG, from both mossy fibers (presynaptic cell) and granule cells(postsynaptic cell), was observed with the same stimulation frequency of 50 Hz. This phenomenon explains the mechanism of synchronized pattern of nerve activity between presynaptic and postsynaptic cells from a molecular viewpoint. Presynaptic signals first activate postsynaptic neurons and then they synchronize between presynaptic and postsynaptic neurons. When postsynaptic cells synchronize with presynaptic cells, the postsynaptic neurons may even initiate further activity by themselves with autocrine mechanism, and enter stage III (FIG. 11). Another possibility is that the mNRG may undergo stimulation-dependent proteolytic cleavage after the exchange of molecular information through a signal from the presynaptic neuron and bi-directional signaling in the course of forming the synapses. In either case, 50 Hz stimulation is thought of as the intermediate stage in communicating between pre- and post-synaptic neurons (FIG. 11, II).

Furthermore, it was revealed the molecular mechanism for NR2C and β2 expressions regulated by sNRG. At low frequency stimulation (1 Hz), the β2 RNA was transcribed more than the NR2C RNA throughout glutamate receptor and ErbB receptor activation. The NR2C mRNA was induced more than β2 at high frequency (100 Hz) accompanying glutamate receptor (particularly NMDA receptor) activation. NR2C and β2 subunit expression could not be observed at 50 Hz which was the optimal frequency for proteolytic cleavage of NRG. It has been already proposed that NR2C expression must require neural activity and that the production efficiency of some soluble forms of NRG might be controlled by electrical activity (Ozaki M, The Neuroscientist, 7(2), 146-154 (2001)). In this Example, it was proved for the first time that proteolytic cleavage of NRG was controlled by patterns of electrical activity in a frequency-dependent manner. Although NRG is necessary to induce NR2C and β2 subunit expressions, discrepancy in optimal value of frequency arose between expression stage (FIG. 11, I and III) and intermediate stage (FIG. 11, II). To explain this discrepancy, pharmacological experiments were performed. Several receptors including the ErbB receptor, have been found to be involved in controlling NR2C and β2 subunit expression from the results of these pharmacological experiments.

Experiments of direct electrical stimulation suggests two new things; (1) Some receptor activation requires endogenous nerve activity to induce gene expression, and (2) direct electrical stimulation can partially rescue the effect of receptor and ion channel blockers (see FIG. 10, C). In the process of synaptic maturation, there may be a cascade between specific patterns of neuronal activity and receptor activation (FIG. 12). If receptor A is activated, neuron will have a pattern A of neuronal activity. Next receptor B is activated by pattern A to produce pattern B. As a result, neurons will have a pattern of A plus B. Each pattern of activity in the cascade may control molecular behavior. It is thought that specific patterns control molecular behaviors and the patterns of neuronal impulses are composed of a combination of activation of individual receptors or channels. Each pattern which was composed contain certain step which controls molecular behavior such as processing of phosphorylated protein. Accordingly the combination of activated receptors and ion channels, and order of their activation may hold the key to solving the discrepancy mentioned above.

In addition, some effects of the receptor blockade by antagonists or blockers could be corrected by specific electrical stimulation. This means that roles of presynaptic neurons, receptor and channel activity can be mimicked by some specific patterns of electrical activity. Accordingly it may be important to examine the role of patterns of electrical activity for artificially controlling neural plasticity.

Effects of the Invention

According to the invention described in claim 1, there is limited damage to cells because the electrodes are not directly inserted into the cells, and it is possible to give electrical stimulation to a large number of cells at the same time.

According to the invention described in claim 2, electrical stimulation can be given at the same time to cells in a plurality of culturing containers by connecting electrode devices to connectors respectively in a condition where the culturing containers are placed on a container supporting means.

Further, according to the invention described in claim 3, a condition is available wherein electricity can be supplied to each connector by connecting the wiring from the changeover device to the adaptor. Furthermore, wiring is placed inside a base frame and therefore an uncluttered wiring structure can be accomplished.

According to the invention described in claim 4, the culturing containers can efficiently be set inside or removed from the base frame by mounting or demounting the container supporting means.

According to the invention described in claim 5, it is possible to give almost even electrical stimulation to cells existing inside the culturing container.

According to the invention described in claim 6 to 9, it is possible to directly and efficiently give electrical stimulation to a large number of nerve cells ex vivo without causing any injuries to the cells.

1. A cell stimulation apparatus for giving electrical stimulation to cultured cells in a non-contact state to the cultured cells, which comprises: a plurality of positive and negative electrodes which extends in an identical direction and are installed on a supporting plate; and supporting means which supports the plural positive and negative electrodes at a position having a predetermined distance from the cultured cells.

2. The cell stimulation apparatus of claim 1, which further comprises container supporting means which supports inside a base frame a culturing container for keeping the cultured cells, and a plurality of connectors connectable to the plural positive and negative electrodes which are provided inside the base frame, wherein the plural connectors are connected to an electrical signal generation device.

3. The cell stimulation apparatus of claim 2 wherein the base frame is provided with an adaptor connectable to the electrical signal generation device and each of the plural connectors is connected to the adaptor by wires inside the base frame.

4. The cell stimulation apparatus of any of claims 1 to 3 wherein the container supporting means is detachable from the base frame.

5. The cell stimulation apparatus of any of claims 1 to 4 wherein the plural positive and negative electrodes are arranged at intervals of 3 to 10 mm.

6. The cell stimulation apparatus of any of claims 1 to 5 wherein the plural electrodes are individually controlled.

7. The cell stimulation apparatus of any of claims 1 to 6 wherein the distance between the cells and the electrodes is kept constant by measuring impedance.

8. A method for electrically stimulating cells, which comprises: setting a plurality of positive and negative electrodes in a culturing plate containing cultured cells, in an identical direction toward a face formed by the cultured cells so that the electrodes do not contact with the cells; forming an electric field using the electrodes; and stimulating the cultured cells by the electric field.

9. The method for electrically stimulating cells according to claim 8 wherein the cultured cells are nerve cells.

10. The method for electrically stimulating cells according to claim 8 or 9 wherein the plural positive and negative electrodes are arranged at intervals of 3 to 10 mm.

11. The method for electrically stimulating cells according to any of claims 8 to 10 wherein the cultured cells are stimulated by staggering the timing of electrical stimulation by means of the plural positive and negative electrodes.

12. The method for electrically stimulating cells according to any of claims 8 to 11 wherein the cells are electrically stimulated using the cell stimulation apparatus of any of claims 1 to 7.


SEQUENCE LISTING (3 sequences)

SEQ ID NO: 1 - protein, 5 aa, Artificial
  Peptide containing C-terminal of secretory neuregulin for use as a hapten
CELYQ

SEQ ID NO: 2 - protein, 6 aa, Artificial
  Peptide containing C-terminal of secretory neuregulin for use as a hapten
CELYQK

SEQ ID NO: 3 - protein, 9 aa, Artificial
  suspected proteolytic cleavage site sequence
ELYQKRVLT